(19) United States
(12) Patent Application Publication
(10) Pub. No.: US 2006/0129240 A1
(43) Pub. Date: Jun. 15, 2006

(54) Implants based on engineered composite materials having enhanced imaging and wear resistance

(76) Inventors: Joe Lessar, Coon Rapids, MN (US); Greg Marik, Germantown, TN (US); Darrel Untereker, Oak Grove, MN (US); Naim Istephanous, Roseville, MN (US); Carlos E. Gil, Collierville, TN (US); Jeffrey P. Rouleau, Maple Grove, MN (US); Robert L. Conta, Mercer Island, WA (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/009,423

(22) Filed: Dec. 10, 2004

(51) Int. Cl. A61F 2/44; A61F 2/30

(52) U.S. Cl. 623/17.14; 623/23.53; 623/23.4; 623/901

(57) ABSTRACT

This invention relates to a metal composite orthopedic device. The device can comprise a metallic substrate cladded or joined to one or more metallic layer(s). The substrate and metallic layer(s) can be selected of different metals and metal alloys to provide desired wear performance, imaging characteristics and optionally to serve as a reservoir for therapeutic agents.

### BACKGROUND OF THE INVENTION

The present invention relates to implantable medical devices formed of metallic, cladded composite materials and to methods of implanting the medical devices into patients in need of treatment. The devices according to the present invention can be used to treat either chronic or acute conditions.

Natural bone joints, for example, joints such as the knees, hips, and intervertebral discs, can be replaced with artificial joints. The artificial joints can be constructed to include ceramic, polymeric, and/or metallic materials. It is important that the artificial joints exhibit good biocompatibility and favorable wear characteristics. Many, but not all, patients undergoing hip or knee replacement are in their sixth decade of life or older. Their joint disorder and/or deterioration can occur because of a chronic condition that has become debilitating, such as osteoarthritis, trauma causing a disruption in the normal joint, or degeneration as a result of the natural aging process. Current artificial joints typically have a useable life span of about 10 to 20 years and will likely perform acceptably for older patients. These devices may not need replacement during the patient's life span. However, younger patients need such devices for longer time frames. The younger patients are also more active. Thus it is not unexpected that implants or replacement joints in younger patients are subjected to greater stress and more motion cycles than those in older patients. Conventional artificial joints may need to be revised after some period of use in younger patients or even in active, older patients. It is desirable that the initial replacement joints survive longer periods of use (up 50 or 60 years) and withstand greater stress to avoid the likelihood of revision and a replacement, which is obviously an undesirable consequence.

It is equally important to minimize any adverse or toxicological problems associated with the production of debris material from wear of the device's articulating surfaces. Consequently, metallic devices are made of wear-resistant, physiologically-acceptable materials such as CoCr alloys.

Some metallic materials may exhibit acceptable wear and biocompatibility characteristics; however, the same materials may also exhibit poor imaging characteristics under commonly-used diagnostic imaging techniques, such as CT and MRI imaging. The imaging characteristics of the implant are important and getting more so. Materials that are highly radiopaque tend to scatter radiation and create artifacts in the image that obscure the peri-prosthetic tissue. This can make it difficult to ascertain the exact location and orientation of the implanted device. The scattered radiation can obscure details of the peri-prosthetic soft and bony tissues that may be important for making regional clinical diagnoses. Additionally, the desired degree of radiopacity (or radiolucency) may vary depending upon the mode of treatment, treatment site, and type of device.

Until now, the selection of materials having appropriate physical and mechanical properties for medical implants has been limited. In general, traditional materials that exhibit good wear characteristics tend to have poor imaging properties. Other materials may have acceptable imaging characteristics but unfavorable wear performance.

Consequently, in light of the above problems, there is a continuing need for advancements in the relevant field including new implant designs, new material compositions, and configurations for use in medical devices. The present invention is such an advancement and provides a variety of additional benefits and advantages.

### BRIEF DESCRIPTION OF THE DRAWINGS

FIG. 1 is a perspective view of one embodiment of a clad, two-piece disc prosthesis in accordance with the present invention.

FIG. 2 is an exploded view of one embodiment of a clad, three-piece disc prosthesis in accordance with the present invention.

FIG. 3 is one embodiment of a clad disc prosthesis assembly in accordance with the present invention.

FIG. 4 is a cross-sectional view of the disc prosthesis assembly of FIG. 3.

FIG. 5 is a perspective view of one embodiment of a cervical spine implant in accordance with the present invention.

FIG. 6 is an exploded view of the spinal implant of FIG. 5.

FIG. 7 is a cross-sectional view of the spinal implant of FIG. 5.

FIG. 8 is a plan view of the lower component of the implant shown in FIG. 5.

FIG. 9 is a perspective view of another embodiment of a spinal implant in accordance with the present invention.

FIG. 10 is a cross sectional view of the spinal implant of FIG. 9.

FIG. 11 is an exploded view of one embodiment of a cervical spine implant in accordance with the present invention.

FIG. 12 is an elevated view of the implant of FIG. 11.

FIG. 13 is a cross-sectional view of the implant of FIG. 11.

FIG. 14 is a cross-sectional view of a cervical spinal implant having a wear-resistant layer secured to a substrate via a mechanical interlocking engagement.

FIG. 15 is a scanned image of a photomicrograph illustrating a cladded material in accordance with the present invention.

FIG. 16 is a scanned image of a photomicrograph of a Ti-6Al-4V substrate material having a layer formed from an ASTM F799 cobalt alloy in accordance with the present invention.

### SUMMARY OF THE INVENTION

The present invention relates to medical implants formed of a material including a “metal matrix composite” the manufacturing and use thereof, and methods of implantation. Various aspects of the invention are novel, nonobvious, and provide various advantages. While the actual nature of the invention covered herein can only be determined with reference to the claims appended hereto, certain forms and features, which are characteristic of the preferred embodiments disclosed herein, are described briefly as follows.

In one form, the present invention provides an orthopedic device that comprises an articulating spinal spacer sized to be inserted into a disc space between adjacent vertebrae. The spinal spacer includes a first member comprising a first layer composed of a first metal and a second layer composed of a different, second metal, and a second member comprising a third layer composed of a third metal and a fourth layer composed of a fourth metal, wherein the first member is configured to engage with the second member to allow a sliding or rotational (or both) movement relative thereto.

In another form, the present invention provides a spinal disc prosthesis. The disc prosthesis includes a first member comprising a first layer composed of a first metal and a second layer composed of a different, second metal, a second member comprising a third layer composed of a third metal and a fourth layer composed of a fourth metal, and an intermediate layer between the first and second member.

In still yet other forms, the present invention provides a method of fabricating an articulating spinal spacer. The method comprises molding a first substrate composed of a first metal, wherein the substrate is sized and configured to be inserted within the space between adjacent vertebrae; and then securing or bonding a second metallic layer to the substrate.

Further objects, features, aspects, forms, advantages and benefits shall become apparent from the description and drawings contained herein.

### DETAILED DESCRIPTION OF THE INVENTION

The present invention includes implantable medical devices that are constructed, or at least partly constructed to include clad materials. In general, the medical devices are formed of a substrate that has been overlaid, inlaid, or through laid with a metal or metal alloy cladding material different than that used in the substrate material. The metallic substrate and the cladding material can be specifically selected and tailored for specific medical applications. The treatment of the materials prior to fabrication, bonding or fabricating techniques to form the clad substrate and/or subsequent treatment can impart beneficial properties to the medical device. This provides greater flexibility to design implantable medical devices with tailored properties. The two materials, the substrate material and the cladding material, can be selected and treated to accomplish two different goals. For example, the one material can be selected for its strength and/or wear resistance, while the other material can be selected for its imaging characteristics. The two materials can then be appropriately combined to provide the implantable medical device that exhibits superior properties.

Specific examples of medical devices that are included within the scope of the present invention include orthopedic implants such as cervical spine implants, intervertebral disc prostheses, vertebral prostheses, bone fixation devices such as bone plates, spinal rods, rod connectors, and drug delivery implants. The medical devices of the present invention can be used to treat a wide variety of animals, particularly vertebrate animals and including humans.

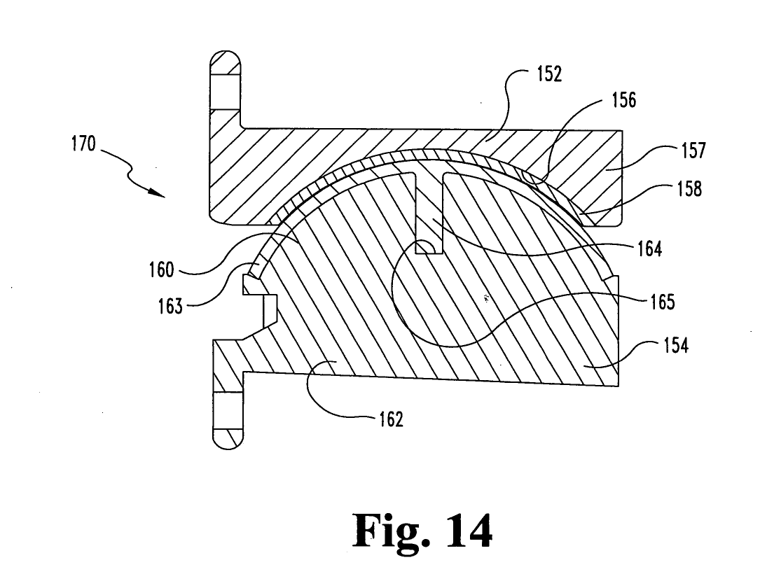

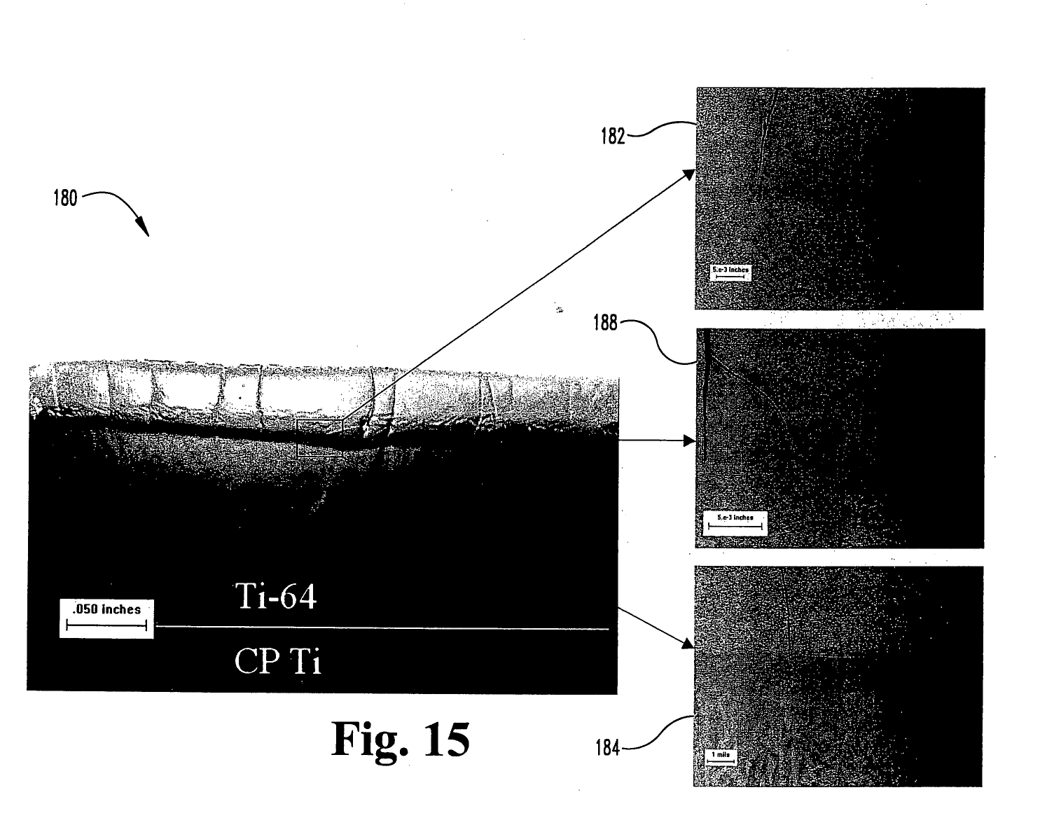

The medical devices based on this invention are formed of a novel composite material construct that includes a metal or metal alloy substrate that is clad, inlaid, or through laid with a second metal or metal alloy. In preferred embodiments, there is no need or requirement for a bonding layer between the metal substrate and the cladding material. However, it will be understood by those skilled in the art that depending upon the method of fabrication, various zones, regions or diffusion layers may exist between the substrate material layer and the cladding layer (see for example FIGS. 14 and 15).

For the present invention, the term “bonding layer” is intended to mean that an intermediate layer, different from either the underlying substrate layer or the cladding layer, is specifically applied—usually in a separate (or sequential) application step.

Preferably, the cladding material is directly bonded, fused, and/or diffused with the metal substrate. These devices can provide particular advantages for use in articulating joints such as spinal implants, disc or nucleus prostheses, which are used to treat spinal disorders. Additionally, the implants of the present invention can be used as joint replacements for joints such as the knee, hip, shoulder, and the like.

The materials for use in the present invention are selected to be biologically and/or pharmacologically compatible. Further, the preferred composites exhibit minimal toxicity, either as part of the bulk device or in particulate or wear debris form. The individual components in the matrix are also pharmacologically compatible. In particularly preferred embodiments, the metallic matrix composite includes at least one component that has been accepted for use by the medical community, particularly the FDA and surgeons.

The substrate and the cladding material for the present invention can be selected from a wide variety of biocompatible metals and metal alloys. Specific examples of biocompatible metals and metal alloys for use in the present invention include titanium and its alloys, zirconium and its alloys, niobium and its alloys, stainless steels, cobalt and its alloys, and mixtures of these materials. In preferred embodiments, the metal matrix composite includes commercially pure titanium metal (CpTi) or a titanium alloy. Examples of titanium alloys for use in the present invention include Ti-6Al-4V. Ti-6Al-6V, Ti-6Al-6V-2Sn, Ti-6Al-2Sn-4Zr-2Mo, Ti-V-2Fe-3Al, Ti-5Al-2.5Sn, and TiNi. These alloys are commercially available in a purity sufficient for the present invention from one or more of the following vendors: ATI Allvac; Timet Industries; Specialty Metals; and Teledyne WaChang. In one embodiment, the materials are specifically selected to provide desired diagnostic imaging characteristics. Preferred materials include pure titanium and titanium alloys such as CpTi and Ti-6Al-4V, respectively. In certain embodiments, the metals and/or metal alloys for use in the present invention do not require any added, dispersed, or encapsulated reinforcing material(s) to provide the desired benefits for orthopedic applications.

The devices of the present invention can be prepared by first forming the substrate material. Thereafter, the cladding material can be overlaid or bonded to the substrate material using a variety of processes to form a laminated or partly laminated device. Preferred processes for forming the substrate include: conventional melting technology, such as, casting directional solidification, liquid injection molding, laser sintering, laser-engineered net shaping, powder metallurgy, metal injection molding (MIM) techniques; and mechanical processes such as rolling, forging, stamping, drawing, and extrusion. The cladding process can include cladding techniques; thermal spray processes include: wire combustion, powder combustion, plasma flame and high velocity Ox/fuel (HVOF) techniques; pressured and sintered physical vapor deposition (PVD); chemical vapor deposition (CVD); or atomic layer deposition (ALD), ion plating and chemical plating techniques.

In selected embodiments, the substrate can comprise a highly-dense metal matrix that can be prepared by a variety of rapid prototyping techniques. Such techniques include conventional melt technology, selective laser sintering, and laser-engineered net shaping (LENS) to name just a few.

Additionally, when desired, the substrate can be porous. Methods for fabricating the porous substrate are described below.

In other embodiments, the substrate for the devices of the present invention can comprise a metallic substrate that can be fabricated using a metal injection molding (MIM) technique. The metal components in powder form and an organic binder can be blended together. The resultant mixture can then be injection molded into a “near net shape” of a desired implant component. This technique can allow for facile fabrication of complex shapes and implant designs that require minimal finishing processes. This technique can provide particular advantages where it is intended to inlay the cladding material into the substrate. The molded article or “green” article can then be subsequently treated using a variety of techniques including CHIP, CIP, HIP, sintering, and densifying as is known in the art.

In yet another embodiment, the substrate for the present invention can be fabricated using powder metallurgy technology either with or without a binder. A binderless powder metallurgy technique can be used to prepare one or more of the components for the devices of the present invention. The binderless powder technique begins with high purity metal powder of controlled morphology and particle size distribution. A master alloy powder of specified chemistry and particle size range, such as 60Al-40V (60% aluminum/40% vanadium) powder, is added to elemental titanium powders to create the Ti-6Al-4V composition. The blend is cold isostatic pressed (CIP) to a density of approximately 85% of theoretical. Vacuum sintering forms the Ti-6Al-4V alloy by diffusion and hot isostatic pressing (HIP) produces the fully dense material and fine-grained microstructure.

For use in the spine, the substrate is fabricated to exhibit suitable strength to withstand the biomechanical stresses and clinically relevant forces without permanent deformation. For devices that are not implanted in the or around the spine, the substrate can be fabricated to withstand the biomechanical forces exerted by the associated musculoskeletal structures. In a preferred embodiment, the substrate is composed of titanium, (CpTi), or a titanium alloy such as Ti-6Al-4V. In this embodiment, the substrate can provide the requisite biomechanical support and still exhibit good diagnostic image characteristics. The substrate can be clad, inlaid, or throughlaid with a cladding material that exhibits good wear characteristics.

The substrate can be clad, inlaid, or throughlaid, or overlaid with a cladding material using thermal spraying techniques. Thermal spray techniques include wire combustion or “metallizing” using a wire material that is fed into to an oxy/fuel gas flame, atomized and then propelled to the target surface. Other thermal spray techniques use a powdered metal composition. A powdered composition is selected to yield the desired cladding material. The powdered composition can be the desired metal or metal alloy or a combination of metal/metal alloys that are combined in the desired amounts. The powdered composition is heated using one of the techniques described below and then sprayed or propelled to the target—the substrate material—where the heated material bonds to the substrate surface. The heating techniques include combustion, plasma flame or plasma spraying and high velocity oxy/fuel HVOP. The thermal spray techniques can provide the advantages of tailored coating properties as desired for specific medical application. For example, a particular material can be sprayed to form a porous material or a dense material. Additionally, the powdered material can be a combination of metals or metal alloys. Subsequent heat treatment and/or mechanical working of the clad substrate can be used to alter the initial microstructure and/or properties as desired.

In one embodiment, it is desirable to provide a substrate that exhibits radiolucent characteristics. In this embodiment, preferred materials include pure titanium metal and titanium alloys. These materials tend to minimize imaging artifacts that can obscure the peri-prosthetic tissues. In other embodiments it is desirable that the substrate exhibits radiopacity. Preferred materials for this embodiment, include cobalt and its alloys and stainless steels.

In other embodiments a porous substrate (and/or a porous clad material) is desired. The pore size can be varied widely depending upon the desired application. For example, the pore size can be selected to allow bone ingrowth into the substrate. In this embodiment, the preferred pore size can be controlled or selected to be between about 50 μm and about 300 μm. More preferably, the pore size can be between about 100 μm and about 200 μm. The pore size as used herein can be determined according to ASTM Standard F1854-01 entitled “Standard Test Method for Stereological Evaluation of Porous Coatings on Medical Implants”.

The pore size can be controlled or selected by varying the constituents of the metal matrix composite. Alternatively, the pore size can be controlled by varying selected process parameters, such as the sintering time, temperature, and pressure. Typically, larger particles induce greater porosity into the matrix. The particle shape can also influence the porosity of the matrix. Generally, particles that do not pack well will increase the porosity of the matrix composite. For example, non-uniform or irregularly shaped particles, particles with a high aspect ratio, or selecting particles from a size distribution will increase the porosity of the matrix composite. Changing the sintering temperature also can impact the porosity of the matrix composite. Increasing the sintering time and/or temperature decreases the porosity.

A porous substrate can also be attained by secondary operations, such as selective dealloying. Pore size and distribution can be tailored by controlling the secondary process parameters.

The pore size can be controlled or selected to facilitate use of the implanted device as a reservoir for one or more therapeutic agents or to facilitate the release of therapeutic agents into adjacent issue. Further, the pore size can be varied and optimized, as desired, to allow a controlled delivery rate for the agents(s); the controlled delivery rate can be for either chronic treatment and/or acute treatment.

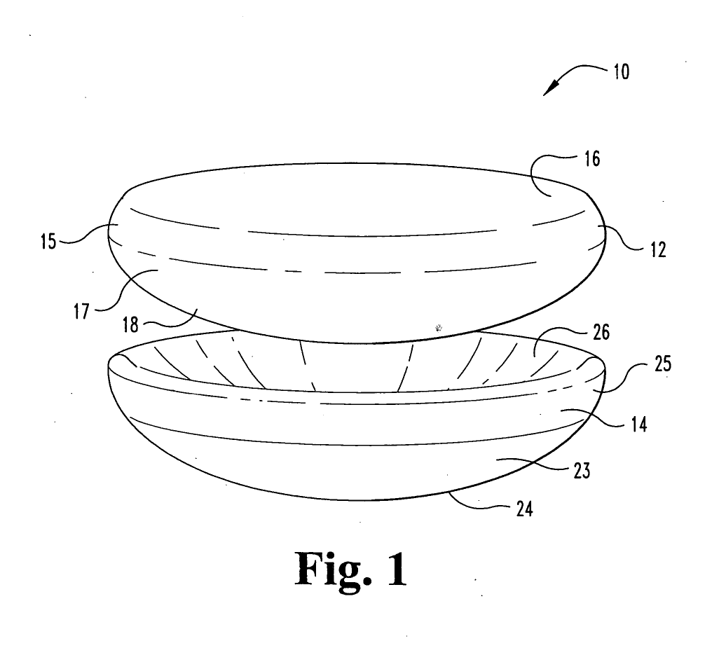

FIG. 1 is an elevated side view of one embodiment of a disc prosthesis 10. Prosthesis 10 is illustrated as comprising two basic components: a first structural member such as a first plate 12, and a second structural member such as a second plate 14. Each of first and second plates are formed of a composite material. First plate 12 comprises a first layer 15 composed of a substrate material and defines a first surface 16 as an upper bone engaging surface. Second layer 17 is composed of a cladding material and defines an opposite a bearing surface 18 that directly overlays the first layer 15. Similarly second plate 14 includes a third layer 23 composed of a cladding material defining a third surface 24 and a fourth layer 25 composed of a cladding material and defines an opposite bearing surface 26.

The substrate material(s) and the cladding material(s) can be different materials. However, in a preferred embodiment, the substrate materials for the first and second plates are the same material; similarly, the cladding materials for the first and second plates are the same material. However, it will be understood that in other embodiments, the substrate material and/or the cladding material for the two plates can be composed of different materials. For example, the prosthesis can include one plate comprising a composite (i.e., two or more materials) articulating on a second plate formed of a single metal or alloy.

In the illustrated embodiment, bearing surface 18 exhibits a convex shape, and bearing surface 26 exhibits a concave shape. In use, when inserted into a disc space between two adjacent vertebrae, bearing surface 18 and bearing surface 26 exhibit a sliding and/or rotating engagement with each other. Consequently, bearing surfaces 18 and 26 are individually shaped to conform to each other.

As noted above, each of surfaces 18 and 26 are composed of a clad material. The clad material can be selected to exhibit enhanced wear characteristics over the substrate material. The clad material can be selected as a metal or metal alloy. In preferred embodiments, surfaces 18 and 26 are characterized as having a minimum surface hardness greater than about 20 Rc; more preferably between greater than about 45 Rc.

The substrate materials can be composed of a material selected to enhance the image capabilities of the prosthesis when examined using common diagnostic imaging techniques, such as, CT, or MRI scanning techniques.

In other embodiments, substrate materials can be formed of a porous metal that exhibits a predetermined, or controlled or selected porosity. The pore size can be varied as desired for use in a particular application. For example, the pore size can be selected to allow bone ingrowth. In this embodiment, the pore size can be controlled or selected to be between about 50 μm and about 300 μm. More preferably, the pore size can be between about 100 μm and about 200 μm as desired for a particular application.

The pore size can also be controlled or selected to facilitate use of the implant as a reservoir for one or more therapeutic agents or to facilitate the release of therapeutic agents into adjacent tissue. Further, the pore size can be varied and optimized, as desired, to allow a controlled delivery rate of the agents(s); the controlled delivery rate can be for either chronic and/or acute treatment.

The first surface 16 and the third surface 24 can be configured to engage with a first, opposing vertebral body endplate (not shown). Each of these surfaces can include a shaped surface portion to matingly conform with and engage with the endplate of the opposing vertebra. In the illustrated embodiment, first surface 16 can be configured to engage with the inferior endplate of a cervical vertebral body, while the third surface can be configured to engage with the superior endplate of the adjacent, lower vertebral body. However, it will be understood that prosthesis 10 can be sized to be inserted between any two articulating vertebrae, for example, thoracic, lumbar, and even between the L5 lumbar and the S1 sacral vertebrae.

In alternative embodiments, first surface 16 and or third surface 24 can either be substantially planar or have a flat surface portion. It will also be understood that the endplate of a particular vertebra can be cut and/or shaped during surgery to receive the disc prosthesis and to securely engage with a planar first surface 16 (or third surface 24).

Each of first surface 16 and third surface 24 can include one or more bone engaging structures on the entire surface or surface portions, to ensure secure attachment to the vertebra. Examples of bone engaging structures include teeth, ridges, grooves, rails, a porous surface layer, coating layer(s) formed of a different metallic material, a polymeric material, or a ceramic material (e.g. hydroxyapatite, and the like).

Prosthesis 10 is illustrated to exhibit a bi-convex, cross-sectional shape. In other embodiments, it will be understood that the shape of prosthesis 10 can be varied to include a wedge shape or a lordotic shape to correct or restore the desired disc space height and/or spinal column orientation. Prosthesis 10 can be provided in a size and a shape to promote the desired therapy to treat the spinal defect. Consequently, prosthesis 10 can be provided in a size to fit between adjacent vertebrae such as the cervical vertebrae, the thoracic vertebrae, the lumbar vertebrae, and the sacral vertebrae. Prosthesis 10 can be sized to extend laterally across the entire surface of the endplate of the opposing vertebrae. More preferably, prosthesis 10 can be sized to extend laterally to bear against the apophyseal ring structure. Prosthesis 10 can extend anterior and posterior across the entire endplate of the opposing vertebrae. In the illustrated embodiment, when viewed from above, prosthesis 10 is configured to resemble a shape with a matching geometry to interface with the opposing endplates of the adjacent vertebrae.

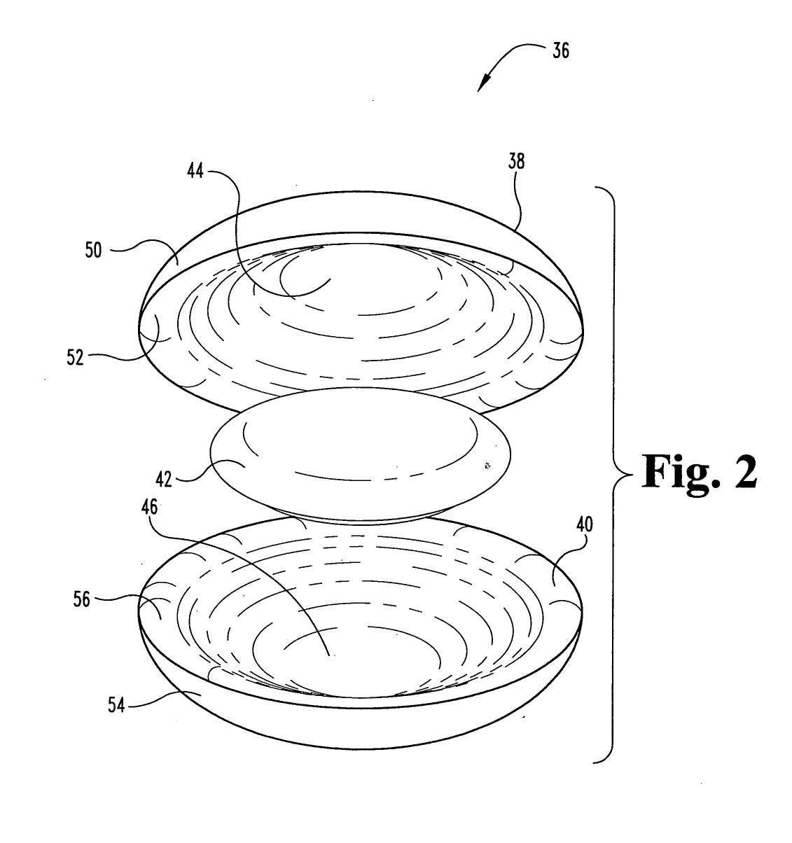

FIG. 2 is an exploded view of an alternative implant assembly 36 in accordance with the present invention. Implant assembly 36 includes an upper structural member, or first plate 38, an opposing, lower structural member, or second plate 40, and an articulating element 42 disposed therebetween. The articulating element engages or rests within a first depression, or recess 44 in first plate 38 and in an opposing depression or second recess 46 in second plate 40.

Both the first plate 38 and second plate 40 are composed of a composite material. First plate 38 comprises a first layer 50 composed of a substrate material and a second layer 52 composed of a cladding material. Similarly, second plate 40 is composed of a third layer 54 composed of a substrate material and a fourth layer 56 composed of cladding material. In a preferred embodiment, second layer 52 directly overlays second layer 50 and fourth layer 56 directly overlays third layer 54. In the illustrated embodiment, second layer 52 is very thin and deposited solely in recess 44 and fourth layer 56 is very thin and deposited solely in recess 46. This can provide particular advantages when the substrate material is selected to be radiolucent such as Ti or a Ti alloy and the cladding material is radiopaque such as CoCr. The resulting prosthesis exhibits good wear characteristics afforded by the thin CoCr wear layer and yet good image characteristics because the CoCr wear layer is surrounded by the more radiolucent material that does not scatter radiation.

Articulating element 42 can be composed of a metallic material, preferably a wear-resistant metal or metal alloy discussed above or more preferably a polymeric material. The polymeric material can be a homogeneous material or a composite material (i.e., an outer shell over an inner core). Articulating element 42 is illustrated as a curved element, preferably having an ovoid shape and/or having a round or oval cross-sectional shape. Alternatively, the articulating element can be provided in a variety of other shapes including spherical, cylindrical or elliptical, disk shape, flattened shape, or wafer and the like.

First and second plates 38 and 40 can be configured similar to second plate 14 of prosthesis 10, including the bone engaging surfaces. Further, first and second plates 38 and 40 approximate mirror images of each other so that recesses 44 and 46 oppose each other when the prosthesis is fully assembled.

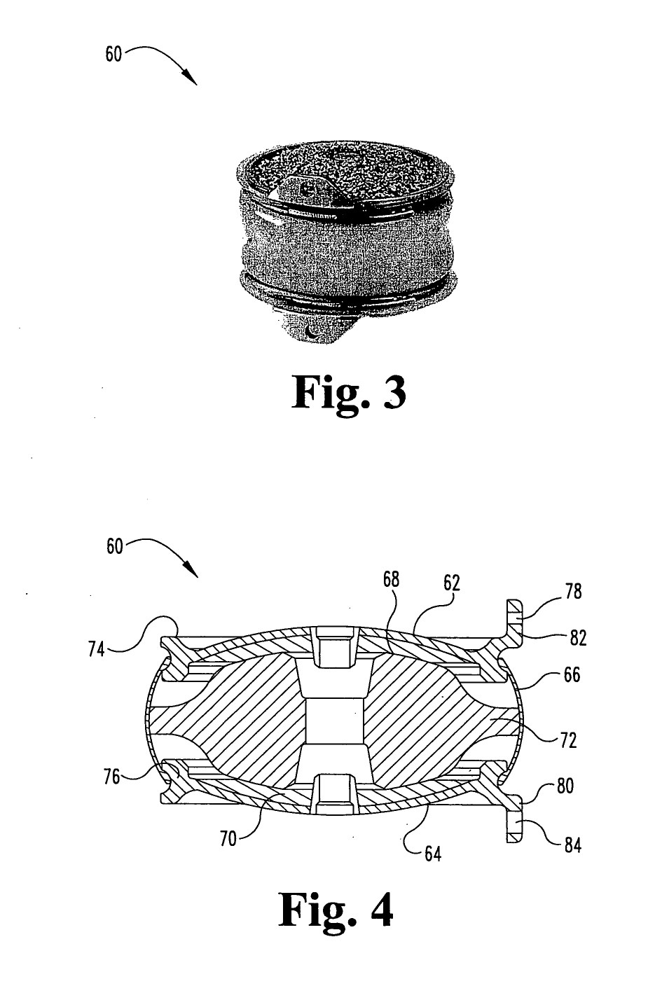

FIG. 3 is a perspective view of one embodiment of a clad disc prosthesis 60 in accordance with the present invention. Disc prosthesis 60 includes a bone engaging first layer 62, an opposite, bone engaging third layer 64, and a peripheral side wall 66 disposed therebetween. Referring additionally to FIG. 4, which is a cross-sectional view of prosthesis 60, first layer 62 can be laminated or bonded to a second layer 68. Similarly, third layer 64 can be bonded or laminated onto a fourth layer 70. Both first layer and third layer can be composed of a first metallic material, and both of second layer 68 and fourth layer 70 can be composed of a different, second metallic material.

In one embodiment, the first metallic material can include a metal or metal alloy selected to provide a porous layer to allow bone ingrowth for fixation of the prosthesis. In addition or in the alternative, each of surfaces 62 and 64 can be fabricated as a porous material that further includes one or more therapeutic agents such as an osteogenic material (including both osteoconductive and osteoinductive materials), an antibacterial agent, antiviral agent, antifungal agent, or a pharmaceutical agent. In one preferred embodiment, bone engaging layers 62 and 64 are formed of a titanium metal or titanium alloy. Examples include commercially pure titanium (CpTi), Ti-Al6-V4, tantalum and its alloys, and niobium and its alloys.

The second metallic material for layers 68 and 70 can be selected to provide the requisite strength needed to withstand the biomechanical forces exerted by the spine. These second and fourth layers can support the bone engaging layers and, consequently, maintain the desired disc space height. The rigid bone engaging surfaces can provide particular advantages in the treatment of patients whose vertebrae—particularly the vertebral endplates—do not provide the strength or support desirable for normal activity because of a degenerative disease or trauma.

Additionally or optionally, an inner core 72 can be positioned between substrate 68 and substrate 70. Inner core 72 can be made out of a suitable biomechanical material such as a polymeric material UHMWPE (ultra high molecular weight polyethylene), a ceramic, a composite, a metal material, and the like. The inner core 72 may be naturally resilient or designed to be resilient such that the prosthesis exhibits an elasticity or stiffness similar to that of a normal, healthy disc. It will be understood that in alternative embodiments, the inner core of prosthesis 60 can be made of a single unitary metallic component or a composite that includes substrate 68, substrate 70, and core material 72.

It will be noted from viewing FIG. 4 in particular, that in the illustrated embodiment layers 62 and 64 are banded by rings 74 and 76, respectively. Each of rings 74 and 76 can be integrally bonded to surfaces 62 and 64. Alternatively, each of rings 74 and 76 can be integrally bonded to substrates 68 and 70. Consequently, in one view, layers 62 and 64 can be considered as an inlaid material into a unitary substrate that includes second layer 68 and ring 74 and fourth layer 70 and ring 76, respectively. Additionally, rings 74 and 76 include a bone engaging feature such as flanges 78 and 80, respectively. Flanges 78 and 80 include through-bores 82 and 84 to aid insertion of the device with a surgical instrument or to provide additional fixation with a bone fixation device such as a bone screw to secure the implant to adjacent vertebral bodies. Spinal prostheses exhibiting similar exterior structures are described in U.S. Pat. Nos. 6,156,067; 6,001,130; 5,865,846; and 5,674,296, each of which is incorporated by reference herein.

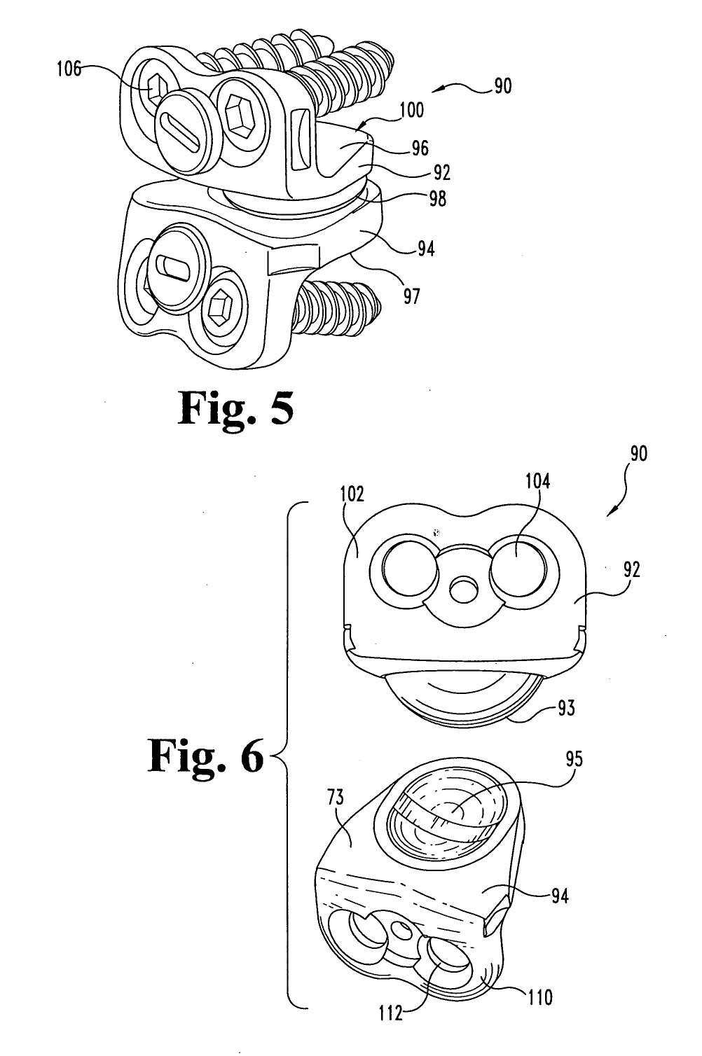

FIG. 5 illustrates an alternative embodiment of a prosthesis or spinal implant 90. Implant 90 includes exterior configurations similar to a prosthesis, which has been previously described in U.S. Pat. Nos. 6,115,637, and 6,540,785, both which are incorporated by reference in their entirety. Implant 90 includes an upper portion 92 and a lower portion 94. Referring additionally to FIG. 6, which is an exploded view of portions of implant 90, upper portion 92 includes a projection 93 that is adapted to be received within a recess 95 formed in lower portion 94. Projection 93 and recess 95 form an articulating couple and allow the upper portion 92 multiaxial motion relative to the lower portion 94.

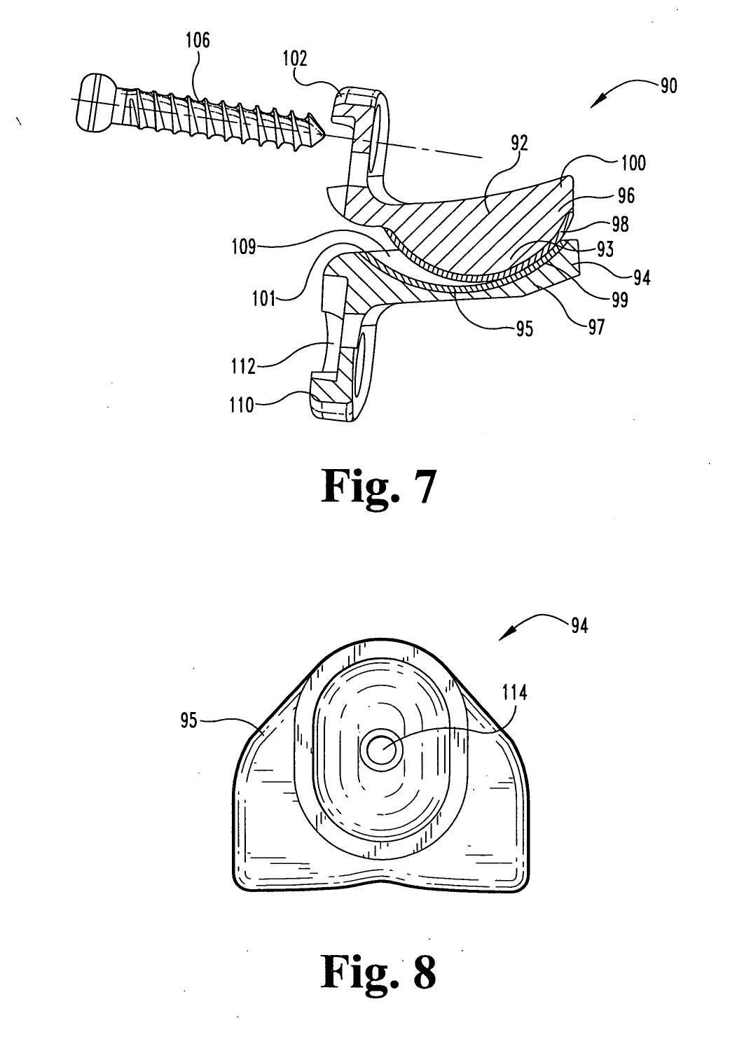

Referring additional to FIG. 7, upper portion 92 is composed of a metallic composite that includes at least two layers. A first layer 96, and a second, wear-resistant, layer 98. The first layer 96 can be formed to include the image-friendly metallic substrate. The second layer is composed of a second metallic material exhibiting suitable wear characteristics. In preferred embodiments the second metallic material exhibits a hardness selected to enhance and extend the useful life span of the implant as it operates or is intended to operate as a disc prosthesis with minimal wear and limited debris loss to the surrounding environment and tissue. This metallic material includes a metal or metal alloy that is compositionally uniform throughout. In particularly preferred embodiments, the second metallic material is composed of a wear-resistant material, for example, a cobalt alloy or stainless steel. Wear-resistant layer 98 and upper surface 96 can be constructed from either the same material or different materials.

Similarly, lower portion 94 includes a clad or layered metal composite having at least a third layer 97 and a fourth layer 99. In the illustrated embodiment, fourth layer defines a trough or inlaid portion 101 for recess 95. Recess 95 is configured to receive or seat projection 93. In one preferred embodiment, recess 95 is configured to allow projection 93 and, consequently, upper portion 92 to rotate or partly rotate about three orthogonal axes and translate or slide, albeit limited, in at least one direction. Preferably recess 95 allows upper portion 92 to slide in the anterior to posterior (AP) direction, referring to the orientation (translation) of the prosthesis in the disc space.

In the illustrated embodiment, upper portion 92 can be configured to include a wide variety of features or structures selected to engage with the endplate of an opposing vertebra. Examples of tissue-engaging structures include teeth, ridges, pores, grooves, roughened surfaces, and wire mesh. As shown in FIG. 7, upper portion 92 can include tissue-engaging structures such as ridge 100. A first flange 102 extends from upper portion 92. Flange 102 can have one, two, or more apertures 104 extending therethrough. Aperture 104 can be a smooth bore or a threaded bore. A bone fastener 106 can be threaded or inserted through aperture 104 and then secured into bone tissue. Bone fastener 106 can be any bone fastener known, described, and/or commonly used for orthopedic applications including screws, staples, wires, pins, rods, sutures, and the like.

Lower portion 94 also can be configured to securely engage with the opposing vertebra and can include tissue engaging structures as has been described above for upper portion 92. Further, lower portion 94 can include a second flange 110 extending therefrom. Second flange 110 can be configured substantially as has been described for first flange 102, including one or more bore or apertures 112 through which bone fasteners can be inserted to engage with underlying tissue.

FIG. 8 is a plan view of lower portion 94 of implant 90 looking down into recess 95. In alternative embodiments, only a portion of recess 95 need be composed of a wear-resistant material. It can be seen in this view that recess 95 includes an inlaid portion formed of a wear-resistant metal or metal alloy. In the illustrated embodiment, the inlay portion is provided as a cylindrical disc positioned at the center of recess 95 and sized to engage the corresponding projection 93. It will be understood that other inlaid shapes are contemplated as included within the scope of the present invention.

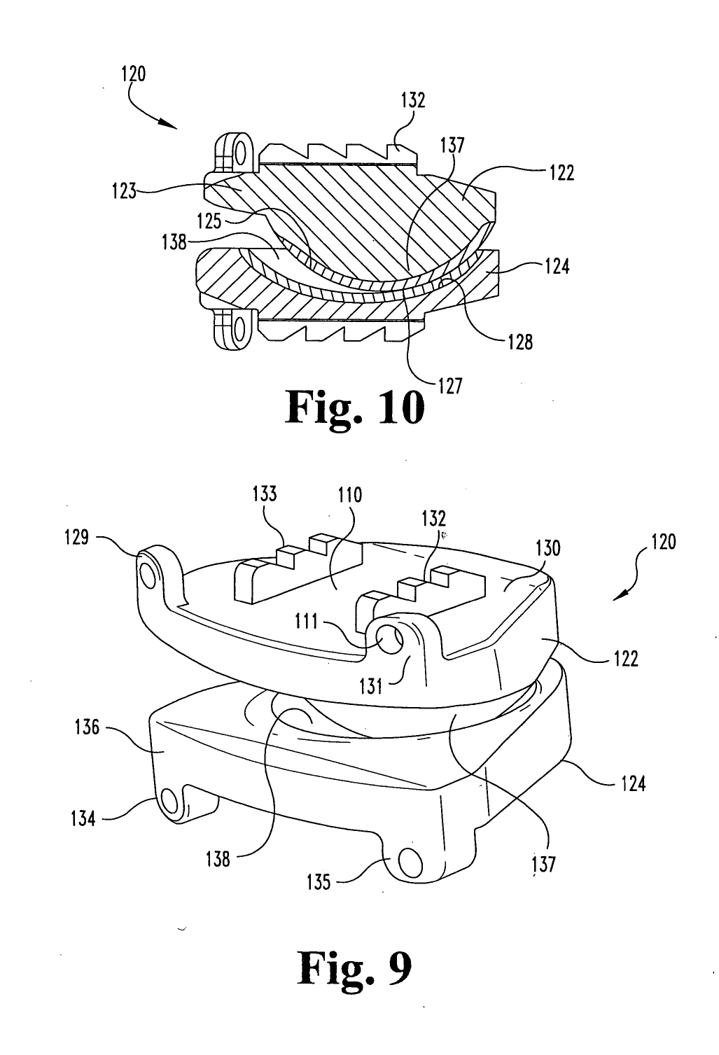

FIG. 9 is a perspective view of yet another embodiment of a spinal implant 120 in accordance with the present invention. Implant 120 is provided as an assembly that includes two basic, separable components: a first or upper portion 122 and a second or lower portion 124. Each of upper portion 122 and lower portion 124 are composed of at least two layers.

Referring additionally to FIG. 10 implant 120 is illustrated in a cross-sectional view. Upper portion 122 includes a substrate material defining a first layer 123 and a second layer 125 composed of a wear-resistant material. Similarly, lower portion 124 includes a substrate material defining a third layer 127 and a fourth layer 128. It can be seen from this view that implant 120 can be provided similar to that which has been described above for implant 50 including a recess or trough 138 formed in lower portion 124. In the illustrated embodiment, trough 138 includes the third layer 127 that is formed of a wear-resistant metal or metal alloy such as has been described above. The remaining bulk of lower portion 124 is provided as substrate or fourth layer 128 and is formed of a material that exhibits good diagnostic imaging characteristics such as titanium or a titanium alloy. Similarly, upper portion 122 and, in particular, protuberance 137, can be cladded or coated with the second layer 125 composed of a wear-resistant material while the bulk or remaining substrate of upper portion 122 can be formed of a material that exhibits acceptable imaging characteristics.

Upper portion 122 can be configured substantially as has been described for upper portion 52 of implant 50. Additionally, upper portion 122 includes two flanges 128 and 129 that are configured to overlay bone tissue. Preferably flanges 129 and 131 are configured to overlay the anterior vertebral body wall portion. Each flange 129 and 131 has at least one bore or aperture through which a surgical instrument or bone fastener can be inserted. Additionally, a first, upper surface 130 includes two rails 132 and 133 extending therefrom. The two rails 132 and 133 each can include teeth or ridges and other surface structures, as noted below, to provide a secure engagement with the opposing endplate of an adjacent vertebra (not shown). In still alternative embodiments, each of rails 132 and 133 can be composed of a material that is different from either the metallic materials of the first and second portions 122 and 124.

Lower portion 124 can be provided substantially as has been described for lower portion 54 of implant 50. Further, lower portion 124 includes two flanges 134 and 135 extending downwardly from an anterior wall 136 (each flange 134 and 135 can include at least one bore or aperture) and the lower surface can include a pair of rails as has been described for the upper portion 122.

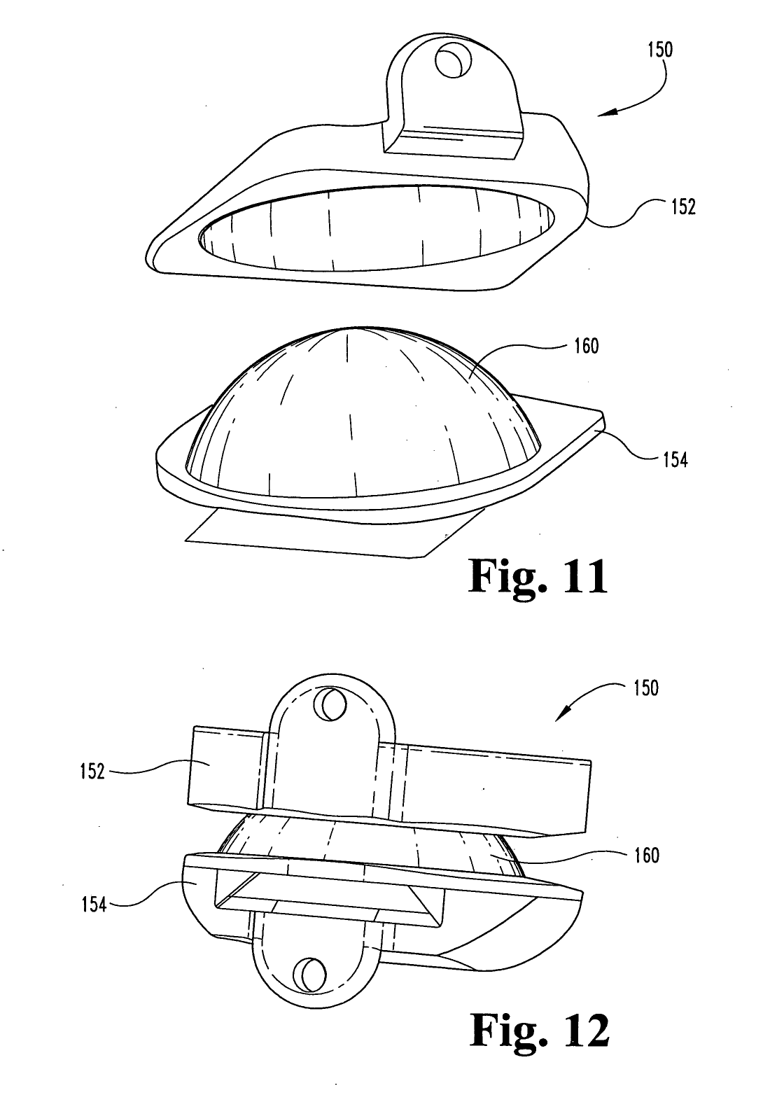

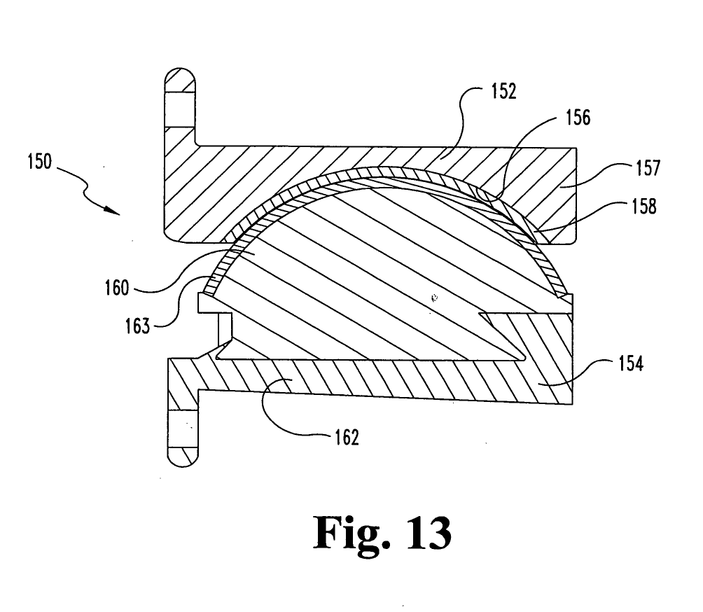

FIGS. 11 through 13 illustrate another embodiment of a disc prosthesis spinal implant 150 provided in accordance with the present invention. Spinal implant 150 includes an upper portion 152 and a lower portion 154. Each of upper portion 152 and lower portion 154 are composed of a composite, layered material. Upper portion 152 includes a first layer 157 and a second layer 158 directly bonded to first layer 157. Upper portion 152 can be provided substantially as has been described above for lower portion 124 of implant 120 and including a recess or trough 156. Trough 156 includes the layer 158 formed of a wear-resistant material. Lower portion 154 includes a protuberance 160, which is bonded or mechanically fixed to a substrate 162. Protuberance 160 is formed of a first metallic material and clad with a second metallic material that forms layer 163. Preferably, layer 163 is a wear-resistant material. Substrate 162 can be the same material as that for protuberance 160 or a different metallic material. Preferably the material(s) for substrate 162 and protuberance 160 is/are selected to provide good diagnostic imaging characteristics and/or permit bone ingrowth. For example, the material for substrate 162 can be selected from titanium or a titanium alloy and can, if desired, include a porous structure to allow bone ingrowth and/or elution of therapeutic agents therefrom.

FIG. 14 illustrates yet another embodiment of a disc prosthesis 170. Prosthesis 170 has a similar exterior configuration as that described for prosthesis 150. Consequently, the same reference numbers will be used to describe like components. In prosthesis 170, substrate 162 and protuberance 160 can be found as a single unitary component. A clad material layer 163 overlays protuberance 160 and is connected via a mechanical interlock arrangement. In the illustrated embodiment, layer 163 includes a pin 164 that is received within recess 165 formed in lower portion 154.

FIG. 15 is a scanned image of a photomicrograph of a composite material including three layers of biocompatible materials including a first, stainless steel layer 182, an intermediate titanium alloy 184 (TI-6Al-4V), and a third layer 186 of a commercially pure titanium material. It can be observed from the scanned image that the stainless steel material provides a diffusion interface 188 between layers 182 and 184. Material 180 is formed by the LENS process that involves melting a powder using a laser. However, other manufacturing process are equally effective and contemplated to be within the scope of the present invention.

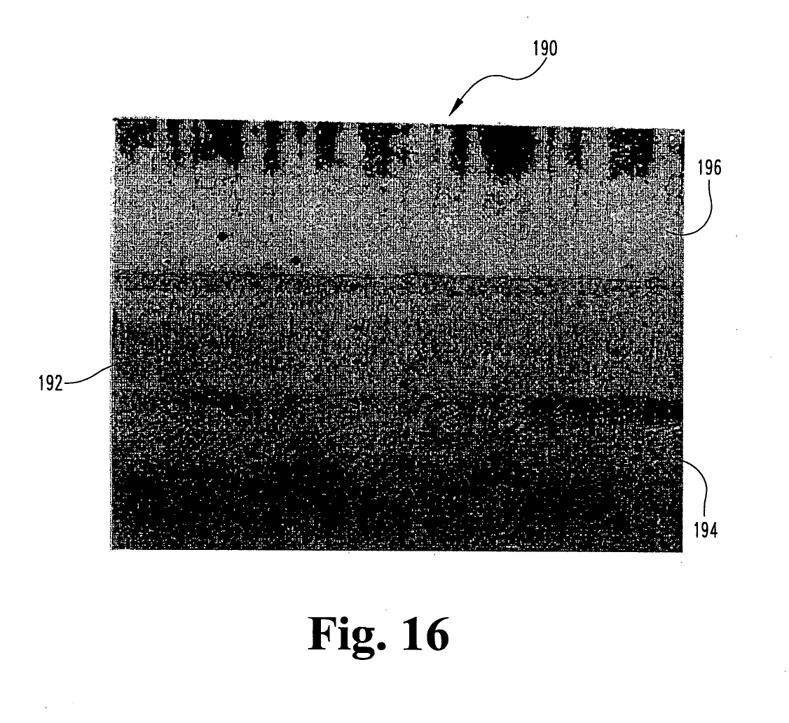

FIG. 16 is a scanned image of a photomicrograph 190 formed of a metallic composite 192 composed of a first layer 194 of a titanium alloy (Ti-6Al-4V) onto which is bonded a second layer 196 of a Co—Cr—Mo alloy referred to ASTM F799.

The present invention contemplates modifications as would occur to those skilled in the art without departing from the spirit of the present invention. In addition, the various procedures, techniques, and operations may be altered, rearranged, substituted, deleted, duplicated, or combined as would occur to those skilled in the art. All publications, patents, and patent applications cited in this specification are herein incorporated by reference as if each individual publication, patent, or patent application was specifically and individually indicated to be incorporated by reference and set forth in its entirety herein.

Any reference to a specific direction, for example, references to up, upper, down, lower, and the like, is to be understood for illustrative purposes only or to better identify or distinguish various components from one another. Any reference to a first or second vertebra or vertebral body is intended to distinguish between two adjacent vertebrae and is not intended to specifically identify the referenced vertebrae as first and second cervical vertebrae or the first and second lumbar, thoracic, or sacral vertebrae. These references are not to be construed as limiting any manner to the medical devices and/or methods as described herein. Also, while various devices implants, and/or portions are described a bilaminates, it will be understood that such devices, implants and portions can include multi-laminates and are intended to be included within the scope of the present invention. Unless specifically identified to the contrary, all terms used herein are used to include their normal and customary terminology. Further, while various embodiments of medical devices having specific components and structures are described and illustrated herein, it is to be understood that any selected embodiment can include one or more of the specific components and/or structures described for another embodiment where possible.

Further, any theory of operation, proof, or finding stated herein is meant to further enhance understanding of the present invention and is not intended to make the scope of the present invention dependent upon such theory, proof, or finding.

What is claimed is:

1. An orthopedic device comprising:
an articulating spinal spacer sized to be inserted into a disc space between adjacent vertebrae, said spacer including:
a first member comprising a first layer composed of a first metal and a second layer composed of a different, second metal, and
a second member comprising a third layer composed of a third metal and a fourth layer composed of a fourth metal, wherein the first member is configured to engage with the second member to allow a sliding and/or rotational movement relative thereto.

2. The device of claim 1 wherein the second layer substantially encases the first layer.

3. The device of claim 1 wherein the first layer is composed of a metal or metal alloy selected from the group consisting of: titanium, titanium-aluminum-vanadium alloy, titanium alloy, zirconium, a zirconium alloy, niobium, and niobium alloys.

4. The device of claim 1 wherein the second layer is composed of a metal or metal alloy selected from the group consisting of: titanium, titanium alloys, cobalt alloys, and stainless steels.

5. The device of claim 1 wherein the second layer is fabricated to exhibit a hardness of at least 20 Rc.

6. The device of claim 1 wherein the first layer and the second layer are directly bonded together.

7. The device of claim 1 wherein first layer is diffusion bonded to the second layer.

8. The device of claim 1 wherein the first metal and the third metal are the same.

9. The device of claim 1 wherein the second and third layer in combination define a wear couple.

10. The device of claim 1 wherein the first layer is porous.

11. The device of claim 10 wherein the first layer comprises a therapeutic agent absorbed within the first layer.

12. The device of claim 11 wherein the therapeutic agent is an osteogenic, osteoconductive, or osteoinductive material.

13. The device of claim 11 wherein the therapeutic agent is an antibiotic, antiviral or antifungal agent.

14. The device of claim 11 wherein the first layer has pores with an average diameter of between about 50 μm and about 300 μm.

15. The device of claim 11 wherein the second layer is nonporous.

16. The device of claim 1 wherein the first member or the second member comprises a fifth layer composed of a metal, ceramic or polymeric material.

17. The device of claim 1 wherein the first layer is nonporous.

18. The device of claim 1 wherein the first member includes a projection clad with the second metal.

19. The device of claim 18 wherein the second member includes a recess configured to receive the projection.

20. The device of claim 19 wherein the recess is inlaid or covered with the fourth metallic layer.

21. The device of claim 1 wherein the second layer defines an inlaid portion in the first layer.

22. The device of claim 21 comprising a plurality of inlaid portions.

23. A spinal disc prosthesis comprising:
a first member comprising a first layer composed of a first metal and a second layer composed of a different, second metal,
a second member comprising a third layer composed of a third metal and a fourth layer composed of a fourth metal, and
an intermediate material layer therebetween.

24. The device of claim 23 wherein the first layer is composed of a metal or metal alloy selected from the group consisting of: titanium, titanium- aluminum-vanadium alloy, titanium alloy, zirconium, a zirconium alloy, niobium, and niobium alloys.

25. The device of claim 23 wherein the second layer is composed of a metal or metal alloy selected from the group consisting of: titanium, titanium alloys, cobalt alloys, and stainless steels.

26. The device of claim 23 wherein the first layer and the second layer are directly bonded together.

27. The device of claim 23 wherein first layer is diffusion bonded to the second layer.

28. The device of claim 23 wherein the first metal and the third metal are composed of the same material.

29. The device of claim 23 wherein the first layer is porous.

30. The device of claim 29 wherein the second layer is porous.

31. The device of claim 23 wherein the first layer comprises a therapeutic agent absorbed therein.

32. The device of claim 31 wherein the therapeutic agent is an osteogenic, osteoconductive, or osteoinductive material.

33. The device of claim 31 wherein the therapeutic agent is an antibiotic, antiviral or antifungal agent.

34. The device of claim 31 wherein the first layer has pores with an average diameter of between about 50 μm and about 300 μm.

35. The device of claim 26 comprising a first surface configured for mating engagement to an inferior vertebral endplate.

36. The device of claim 35 comprising a second surface configured for mating engagement to a superior vertebral endplate.

37. A method of fabricating an articulating spinal spacer; said method comprising:
molding a first substrate composed of a first metal, said substrate sized and configured to be inserted within a disc space between adjacent vertebrae; and
securing a metallic layer to the substrate.

38. The method of claim 37 wherein said molding comprises laser sintering a metallic composition.

39. The method of claim 37 wherein said molding comprises laser- engineered net shaping

40. The method of claim 37 wherein said molding comprises metal injection molding techniques.

41. The method of claim 37 wherein said bonding comprises using thermal spray processes.

42. The method of claim 37 wherein said bonding comprises using wire combustion techniques.

43. The method of claim 37 wherein said bonding comprises using powder combustion techniques.

44. The method of claim 37 wherein said bonding comprises using plasma flame or a high velocity Ox/fuel (HVOF) techniques

45. The method of claim 37 wherein said bonding comprises using physical vapor deposition, chemical vapor deposition, or atomic layer deposition techniques.

46. The method of claim 37 wherein the clad layer and the substrate are mechanically joined together.
